(19) United States
(12) Patent Application Publication
(10) Pub. No.: US 2010/0330158 A1
(43) Pub. Date: Dec. 30, 2010

(54) PROTEIN-ASSISTED DRUG DELIVERY SYSTEM FOR THE TARGETED ADMINISTRATION OF ACTIVE AGENTS

(75) Inventors: Vishal Soni, Dayton, NJ (US); Kanaiyalal R. Patel, Union, NJ (US); Dasaradhi Lakkaraju, Princeton Jct., NJ (US); Navneet Puri, Bridgewater, NJ (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: InnoPharma, LLC, Monmouth Junction, NJ (US)

(21) Appl. No.: 12/748,221

(22) Filed: Mar. 26, 2010

Related U.S. Application Data
(60) Provisional application No. 61/165,416, filed on Mar. 31, 2009.

(51) Int. Cl. A61K 9/127; A61K 38/00; A61P 25/00

(52) U.S. Cl. 424/450; 514/1.2

(57) ABSTRACT

The present invention provides a composition and prodrug for targeted drug delivery to the central nervous system of a patient. The inventive composition and prodrug include a pharmaceutically acceptable active agent and at least one protein selected from the group consisting of a fimbrial adhesin protein, a membrane protein, and combinations thereof. The inventive compositions and prodrugs of the present invention selectively target the blood-brain barrier and deliver hydrophilic and lipophilic active agents of varying sizes to the central nervous system.

### CROSS-REFERENCE TO RELATED APPLICATIONS

This patent application claims the benefit of U.S. Provisional Patent Application No. 61/165,416, filed Mar. 31, 2009, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 9,556 Byte ASCII (Text) file named “268643_SEQUENCE_LISTING_{—}03-26-10.TXT,” created on Mar. 5, 2010.

### FIELD OF THE INVENTION

The present invention relates to a system for targeted drug delivery of hydrophilic and lipophilic active agents of varying sizes to the central nervous system (CNS) of a patient by enabling the active agent to cross the blood-brain barrier (BBB). The present invention also relates to methods for the preparation of the drug delivery system and methods of treatment using the drug delivery system. The drug delivery system of the present invention enables efficient administration of active agents to the CNS.

### BACKGROUND OF THE INVENTION

Despite the worldwide prevalence of CNS associated diseases, only a small number of pharmaceutical products have been developed which effectively treat CNS afflictions. The principle reason for this under-development is that the great majority of drug candidates are not able to successfully reach and cross the brain capillary wall, which forms the CNS-protecting BBB in vivo.

The BBB is formed by high-density endothelial cells packed together through tight junctions. Star shaped glial cells called astrocytes provide biochemical support to endothelial cells and assist in such tight packing. Very tight packing at the endothelia in brain capillaries restricts the passage of most solutes and bigger lipophilic molecules from blood to neural tissue. Given the capillary wall size restrictions, only very small lipophilic molecules with a molecular mass less than approximately 400-500 Daltons (Da) can effectively cross the BBB via the passive diffusion mechanism. In this regard, larger and less lipophilic molecules are usually blocked from the CNS by the BBB and are metabolized and excreted by the body before they can give rise to any CNS associated activity. Unfortunately, there are only a few diseases of the CNS that consistently respond to small lipophilic molecules which can cross the BBB via passive diffusion. Indeed, many serious disorders of the CNS do not respond to conventional, lipid-soluble, low molecular weight, small-molecule therapeutics.

One approach for delivering less lipophilic drugs across the BBB is to increase the lipophilicity of the drug. A drug can be lipidated either by masking polar functional groups with lipophilic moieties or by conjugating the drug to a lipid-soluble drug carrier. Conjugation to a lipid-soluble drug carrier results in the production of a lipophilic prodrug which can cross the BBB. Once across the BBB, the prodrug is then metabolized within the CNS and converted to the parent drug, which is then able to provide the desired therapeutic effect.

While effective in some instances, lipidation of drugs has a number of limitations. First, lipidation not only increases the lipophilicity of the active, but also increases the size. As discussed above, only smaller lipophilic drugs can effectively cross the BBB via passive diffusion. Accordingly, the ability of a drug to permeate the BBB decreases exponentially as the molecular size of the drug increases. Thus, an increase in drug size may adversely affect the transfer of the drug across the BBB. Secondly, increased lipophilicity also increases the drug penetration in other organs of the body. This can lead to a decreased blood half-life of the drug, a reduction in the drug plasma concentration as measured by area under the curve (AUC), and, ultimately, an increase in unwanted side effects.

Another approach for delivering larger, less lipophilic drugs across the BBB is to exploit one of the different classes of BBB catalyzed transport mechanisms. These mechanisms are intrinsic to the BBB and are necessary to actively transport various essential elements (e.g., minerals, nutrients, etc.) from the blood across the BBB to neuronal tissues. The BBB transport systems are situated on the luminal and abluminal membranes of the brain capillary endothelium and are each specific for a particular essential element. For example, the transferrin receptor is involved in the transport of iron across various membranes including the BBB. Similarly, Glut1 transporter is expressed in the capillary endothelium of the human brain to shuttle glucose across the BBB.

A vast amount of research has been directed to using the two main classes of endogenous transport mechanisms (e.g., carrier- and receptor-mediated transport) to deliver drugs through the BBB to the CNS. In this regard, a limited number of successful drug delivery strategies have been developed. For example, Type 1 Large Neutral Amino Acid Transporter (LAT1) has been used to transport the α-amino acid form of dopamine (i.e., LDOPA) to the brain in an effort to treat Parkinson's disease. However, as a general rule, the BBB transport system is not specific for drugs. Thus, binding specificity is often an issue during attempts to utilize the endogenous transport mechanisms for drug delivery and, as a result, increased side effects are often implicated in such studies.

Accordingly, there remains a need for drug delivery systems for hydrophilic and lipophilic drugs of any size which selectively target the BBB.

### BRIEF SUMMARY OF THE INVENTION

The present invention provides a composition for targeted drug delivery to the CNS of a patient and methods for the preparation of the targeted drug delivery composition. The inventive composition includes a pharmaceutically acceptable active agent, at least one protein selected from the group consisting of a fimbrial adhesin protein, a membrane protein, and combinations thereof, and a pharmaceutically acceptable carrier. In a preferred embodiment, the composition contains a fimbrial adhesion protein selected from the group consisting of S fimbriae, variants of S fimbriae, and combinations thereof. In another preferred embodiment, the composition contains a membrane protein selected from the group consisting of outer membrane protein A (OmpA), variants of OmpA, and combinations thereof.

The inventive compositions and prodrugs of the present invention selectively target the BBB and deliver hydrophilic and lipophilic active agents of varying sizes to the CNS. As such, the inventive compositions and prodrugs of the present invention show increased BBB permeability that may lead to increased therapeutic efficacy over that observed with presently available non-targeted compositions containing related active agents. In this regard, the present invention also provides methods of delivering an active agent to the central nervous system of a patient in need thereof by administering the inventive compositions.

These and other advantages of the present invention, as well as additional inventive features, will be apparent from the description of the invention provided herein.

### DETAILED DESCRIPTION OF THE INVENTION

The present invention is directed to a composition for targeted drug delivery to the CNS of a patient comprising a pharmaceutically acceptable active agent, at least one fimbrial adhesin protein and/or at least one membrane protein, and a pharmaceutically acceptable carrier. As used herein, the CNS refers to the parts of the nervous system that function to coordinate the activity of all systems in the body of a vertebrate organism. More specifically, the CNS refers to the brain and spinal cord of a vertebrate organism, wherein the brain and spinal cord are enclosed in the meninges. In a preferred embodiment, the CNS is any neural tissue protected by the BBB.

As used herein, the patient is any vertebrate organism in need of active agent therapy. Preferably, the patient is a mammalian host. More preferably, the patient is a human.

The protein for use in the inventive composition can be any suitable protein provided the protein assists in active agent delivery to the BBB and/or active agent penetration of the BBB. Suitable proteins for use in the inventive composition can be isolated from a pathogenic bacteria or virus. Exemplary organisms which produce suitable proteins include, for example, Escherichia spp. (e.g., E. coli), Pseudomonas spp. (e.g., P. aeruginosa), Klebsiella spp. (e.g., K. pneumonia), and Salmonella spp. (e.g., S. choleraesuis). Preferably, the protein employed in the inventive composition is isolated from E. coli. More preferably, the E. coli is an E. coli K1 strain, an E. coli K1-subtype strain, an E. coli CFT073 strain, or an E. coli 0157:H7 strain.

Pathogenic E. coli K1, E. coli K1-subtypes, E. coli CFT073, or E. coli 0157:H7 express many types of fimbrial adhesins and membrane proteins. For example, E. coli K1, E. coli K1-subtypes, E. coli CFT073, or E. coli 0157:H7 express, among others, Type 1 fimbrial adhesins, P-Type fimbrial adhesins, S-Type fimbrial adhesins, and multiple types of outer membrane proteins. In this regard, suitable proteins for use in the inventive composition demonstrate affinity for human brain microvascular endothelial cells (HBMEC).

In one embodiment, suitable proteins for use in the inventive composition are fimbrial adhesin proteins and/or membrane proteins isolated from E. coli K1, E. coli K1-subtypes, E. coli CFT073, or E. coli 0157:H7. In a preferred embodiment of the present invention, the fimbrial adhesin protein is S fimbriae isolated from E. coli K1, E. coli K1-subtypes or E. coli CFT073. In another preferred embodiment of the present invention, the membrane protein is OmpA isolated from E. coli K1, E. coli K1-subtypes or E. coli 0157:H7. In this regard, the membrane protein may comprise, consist, or consist essentially of SEQ ID NO: 6 (corresponding to ompA isolated from E. coli 0157:H7). In yet another preferred embodiment, the composition of the present invention comprises a combination of S fimbriae isolated from E. coli K1, E. coli K1-subtypes or E. coli CFT073 and OmpA isolated from E. coli K1, E. coli K1-subtypes or E. coli 0157:H7.

In certain instances, fimbrial adhesin proteins and membrane proteins can elicit systematic immunogenic responses when administered to a patient. However, the immunogenic characteristics of the fimbrial adhesin and/or membrane protein material do not necessarily reside in the complete protein structure. Accordingly, another embodiment of the present invention includes a composition comprising a variant of a fimbrial adhesin protein and/or membrane protein wherein the HBMEC affinity is maintained but the protein is changed so as to minimize any immunogenic response.

As used herein, the term “variant” includes homologues as well conservative and/or non-conservative alterations of the polypeptide sequence of the native protein. The term “variant” also refers to synthetic equivalents to the native protein. In some embodiments, a variant includes one or more amino acid substitutions, insertions, and/or deletions compared to the protein from which it was derived, and yet retains its respective activity. For example, a variant can retain at least about 10% of the biological activity of the parent protein from which it was derived, or alternatively, at least about 20%, at least about 30%, or at least about 40% of the biological activity of the parent protein. In some preferred embodiments, a variant retains at least about 50% of the biological activity of the parent protein from which it was derived. In another embodiment, the substitutions, insertions, and/or deletions may result in enhanced biological activity when compared to the parent protein. For example, the variant may have a biological activity of at least about 100% of the biological activity of the parent protein from which it was derived, or alternatively, at least about 110%, at least about 120%, at least about 150%, at least about 200%, or at least about 1000% of the biological activity of the parent protein from which it was derived.

In other embodiments, the fimbrial adhesin protein and/or membrane protein variant incorporated in the composition of the present invention has a polypeptide sequence having at least about 10% (e.g., at least about 15%, at least about 20%, at least about 25%, at least about 30%, at least about 35%, at least about 40%, at least about 45%, at least about 50%, at least about 60%, at least about 70%, at least about 80%, at least about 85%, at least about 90%, at least about 95%, at least about 96%, at least about 97%, at least about 98%, at least about 99%, or at least about 99.5%) sequence identity with the fimbrial adhesin protein and/or membrane protein from which it was derived.

The amino acid substitutions, insertions, and/or deletions of a variant can occur in any domain of the protein. In a similar manner, the corresponding variant may possess additional functional domains or an absence of functional domains compared to the protein from which it was derived. Most preferably, the variant is a polypeptide fragment of the protein which maintains the functional domain or domains of the native protein involved in CNS delivery. In this regard, the variant may comprise, consist, or consist essentially of SEQ ID NO: 2 (corresponding to sfaS isolated from E. coli CFT073 with 22 amino acids removed from the 5′ end and a methionine added to the 5′ end as compared to the wild type sfaS amino acid sequence) or SEQ ID NO: 4 (corresponding to sfaA isolated from E. coli CFT073 with 30 amino acids removed from the 5′ end and a methionine added to the 5′ end as compared to the wild type sfaA amino acid sequence). The functional domain which is maintained in the variant can be any suitable functional domain. In a preferred embodiment, the functional domain is a binding domain or a domain involved in BBB penetration.

The fimbrial adhesin proteins and/or membrane proteins and variants thereof employed in the compositions of the present invention can be isolated from a pathogenic bacteria or virus and purified by any suitable methods known to those of skill in the art. Similarly, when necessary, the fimbrial adhesin proteins and/or membrane proteins and variants thereof can be produced either by using recombinant technology or synthesized and purified by any suitable methods known to those of skill in the art.

The inventive protein-assisted composition of the present invention can be used to administer virtually any pharmaceutically acceptable active agent. Suitable active agents for use in the inventive compositions include both hydrophilic and lipophilic active agents. In a similar manner, active agents of varying molecular weight can be employed in the compositions. In one embodiment, suitable active agents can have a molecular weight of less than about 500 Da. For example, exemplary active agents can have a molecular weight of less than about 400 Da, less than about 300 Da, less than about 200 Da, or less than about 100 Da. In another embodiment, suitable active agents can have a molecular weight of greater than about 500 Da. For example, exemplary active agents can have a molecular weight of greater than about 600 Da, greater than about 700 Da, greater than about 800 Da, greater than about 900 Da, or greater than about 1000 Da.

Preferable active agents for use in the inventive compositions are capable of inducing (either directly or indirectly) a CNS associated therapeutic effect when transported through the BBB to the CNS. In this regard, suitable active agents can, for example, provide treatment for Alzheimer's disease, Parkinson's disease, brain cancer, stroke, brain injury, spinal cord injury, HIV infection of the brain, an ataxia-producing disorder, amyotrophic lateral sclerosis, Huntington's disease, multiple sclerosis, affective disorders, anxiety disorders, epilepsy, meningitis, neuromyelitis optica, late-stage neurological trypanosomiasis, progressive multifocal leukoencephalopathy, De Vivo disease, depression, chronic pain, or a childhood inborn genetic error affecting the brain. Additional examples of active agents for use in the present invention include antineoplastics, antidepressants, anti-inflammatory, antipsychotics, analgesics, and sedatives.

In one embodiment, the present invention is directed to a composition for targeted drug delivery to the CNS of a patient comprising an active agent and at least one fimbrial adhesin protein and/or membrane protein, wherein the composition further comprises a pharmaceutically acceptable carrier. Exemplary pharmaceutically acceptable carriers include, for example, excipients, binders, disintegrants, corrigents, flavors, emulsifiers, solvents, diluents, dissolution aids, and the like. The pharmaceutically acceptable carrier may be a mixture of one or more pharmaceutically acceptable carriers. In a preferred embodiment, the pharmaceutically acceptable carrier is a pH stabilized solution. The composition of the present invention can be formulated according to any of the conventional methods known to those of skill in the art.

In a further embodiment, the present invention is directed to a composition for targeted drug delivery to the CNS of a patient comprising an active agent and at least one fimbrial adhesin protein and/or membrane protein, wherein the composition is a liposomal composition. More specifically, the liposomes comprise a lipophilic portion comprising a membrane forming lipid. Liposomes are well known in the art as spherical drug-delivery vesicles composed of at least one lipid bilayer membrane surrounding an internal aqueous cavity. In the case of the present invention, the liposomes can comprise one (unilamellar vesicles) or more (multilamellar vesicles) lipid bilayer membranes depending upon the particular composition and procedure used to make them.

The targeted drug delivery liposomes of the present invention can have any suitable mean particle size. In one embodiment, the liposomes of the present invention have a mean particle diameter of up to and including about 1000 microns. In a further embodiment, the liposomes of the present invention have a mean particle diameter of about 0.005 microns to about 500 microns. Preferably, the liposomes have a mean particle diameter of about 0.005 microns to about 50 microns. More preferably, the liposomes have a mean particle diameter of about 0.005 microns to about 5 microns. Even more preferably, the liposomes have a mean particle diameter of about 0.005 microns to about 0.5 microns.

The liposomal composition of the present invention can contain any suitable amount of active agent. The liposomal composition can further contain a targeting ligand in any suitable amount. In one embodiment, the targeting ligand may be least one fimbrial adhesion protein selected from the group consisting of S fimbriae, variants of S fimbriae, and combinations thereof. In another embodiment, the targeting ligand may be a membrane protein selected from the group consisting of OmpA, variants of OmpA, and combinations thereof.

The protein-assisted active agent-encapsulated liposomes of the present invention comprise a lipophilic portion comprising at least one membrane forming lipid. The membrane forming lipid for use in the inventive liposomal compositions can be any suitable membrane forming lipid. Suitable membrane forming lipids include pharmaceutically acceptable synthetic, semi-synthetic (modified natural), or naturally occurring compounds having a hydrophilic region and a hydrophobic region. Such compounds include amphiphilic molecules which can have net positive, negative, or neutral charges or which are devoid of charge. Accordingly, the active agent-encapsulated liposomes of the present invention can be positively charged, negatively charged, or neutral. A mixture of membrane forming lipids may also be used.

In one embodiment, a portion of the lipid is derivatized by a hydrophilic polymer to provide a hydrophilic polymer-derivatized lipid. The hydrophilic polymer can be any suitable hydrophilic polymer. Suitable hydrophilic polymers include, for example, polyethylene glycol (PEG), polyvinylpyrrolidone (PVP), polyvinylmethylether, polymethyloxazoline, polyethyloxazoline, polyhydroxypropyloxazoline, polymethacrylamide, polydimethacrylamide, polyhydroxypropylmethacrylamide, polyhydroxypropylmethacrylate, polyhydroxyethylacrylate, hydroxymethylcellulose, hydroxyethylcellulose, polyaspartamide, and polysaccharide. In a preferred embodiment, the hydrophilic polymer comprises PEG.

The hydrophilic polymer may have any suitable molecular weight. In one embodiment, the hydrophilic polymer has a molecular weight in the range from about 100 Daltons to about 100,000 Daltons. Preferably, the hydrophilic polymer has a molecular weight in the range from about 100 Daltons to about 10,000 Daltons. More preferably, the hydrophilic polymer has a molecular weight in the range from about 500 Daltons to about 5,000 Daltons. In a particularly preferred embodiment, the hydrophilic polymer is PEG having a molecular weight in the range from about 100 Daltons to about 10,000 Daltons.

In one embodiment, a portion of the hydrophilic polymer-derivatized lipid is functionalized to attach to the targeting ligand to provide a functionalized hydrophilic polymer-derivatized lipid. The hydrophilic polymer-derivatized lipid may be functionalized to have any functional group suitable to attach to the targeting ligand. Exemplary functional groups include —OH, —CHO, —COOH, —SH, —NHS, —NHCO, —NHCS, —NH₂, -maleimide, -isocyanate, -hydrazide, -vinylsulfone, and -epoxide. In a preferred embodiment, the functionalized hydrophilic polymer-derivatized lipid is a compound having the formula:

membrane forming lipid-PEG-X,

wherein X is a functional group selected from the group consisting of —OH, —CHO, —COOH, —SH, —NHS, —NHCO, —NHCS, —NH₂, -maleimide, -isocyanate, -hydrazide, -vinylsulfone, and -epoxide.

In one embodiment, the targeting ligand is attached (e.g., covalently bound) to the functionalized hydrophilic polymer-derivatized lipid. In a preferred embodiment, the targeting ligand is attached to the distal end of the functionalized hydrophilic polymer-derivatized lipid. In an especially preferred embodiment, the targeting ligand is attached to the distal end of the functionalized hydrophilic polymer-derivatized lipid having the formula set forth above.

In one embodiment, the targeting ligand is derivatized to generate a functional group. The functional group may be any functional group suitable to attach the targeting ligand to the functionalized hydrophilic polymer-derivatized lipid. For example, the functional group of the targeting ligand may be —OH, —COOH, —SH, or —NH₂.

In one embodiment, the membrane forming lipid is a cationic lipid. The cationic lipid can be any suitable cationic lipid which carries a net positive charge at physiological pH. Preferably, the cationic lipid is effective to impart a positive surface charge to the liposomes. A positive surface charge is believed to enhance the binding of the liposomes to target cells (e.g., the cells of the CNS). Exemplary cationic lipids include N,N-dioleyl-N,Ndimethylammonium chloride (“DODAC”), N-(2,3-dioleyloxy) propyl-N,N-N-triethylammonium chloride (“DOTMA”), N,N-distearyl-N,N-imethylammonium bromide (“DDAB”), N-(2,3-dioleoyloxy) propyl)-N,N,N-trimethylammonium chloride (“DOTAP”), 3-(N—(N′,N′-dimethylaminoethane)(carbamoyl)cholesterol (“DC-Chol”), N-(1-(2,3-dioleyloxy)propyl)-N-2-sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N-(1,2-dimyristyloxyprop-3-yl)-N,N-dimethyl-N-hydroxyethyl ammonium bromide (“DMRIE”), and combinations thereof.

Additionally, a number of commercial preparations of cationic lipids can be used. Exemplary cationic preparations include LIPOFECTIN (including DOTMA and DOPE, available from GIBCO/BRL), LIPOFECTAMINE (comprising DOSPA and DOPE, available from GIBCO/BRL), and TRANSFECTAM (comprising DOGS, in ethanol, from Promega Corp.).

In one embodiment, the membrane forming lipid is an anionic lipid. The anionic lipid can be any suitable anionic lipid. For example, anionic lipids suitable for use in the present invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, anionic modifying groups joined to neutral lipids, and combinations thereof.

In yet another embodiment, the membrane forming lipid is a neutral lipid. The neutral lipid can be any suitable neutral lipid which exist either in an uncharged or neutral zwitterionic form at physiological pH. Exemplary neutral lipids include diacylphosphatidylcholine, diacylphosphatidylethanolamine, ceramide, sphingomyelin, cephalin, cholesterol, cerebrosides, diacylglycerols, and combinations thereof.

In one embodiment, the membrane forming lipid can include fatty acid compounds which contain a hydrocarbon chain linked to a carboxylic acid or ester. The fatty acid compounds can be synthetic or derived from natural sources, such as egg or soy. In addition, the fatty acid compounds for use in the present invention can include fatty acid chains of varying length and saturation. For example, the length of the fatty acid hydrocarbon chain can range from about 4 to about 30 carbon atoms. More preferably, the hydrocarbon chain can range from about 12 to about 24 carbon atoms.

In a preferred embodiment, the membrane forming lipid can include phospholipids which contain a diglyceride moiety and a phosphate group. The phospholipids can be synthetic or derived from natural sources, such as egg or soy. In addition, the phospholipids for use in the present invention can include phospholipids with mixed hydrocarbon chains or singularly pure hydrocarbon chains. The hydrocarbon chains of suitable phospholipids can include chains of varying length and saturation. For example, the length of the hydrocarbon chains can range from about 4 to about 30 carbon atoms. More preferably the hydrocarbon chains can range from about 12 to about 24 carbon atoms.

In one embodiment of the present invention, the membrane forming lipid is an unsaturated phospholipid, a saturated phospholipid, or combinations thereof.

The unsaturated phospholipid can be any suitable unsaturated phospholipid. Exemplary unsaturated phospholipids for use in the lipophilic active agent-encapsulated liposomes of the present invention include egg lecithin, soya lecithin, phosphatidylcholine, dioleoylphosphatidylcholine, diarachidonoylphosphatidylcholine, dilinoleoylphosphatidylcholine, phosphatidylethanolamine, dioleoylphosphatidylethanolamine, egg cephalin, soya cephalin, phosphatidylserine, dioleoylphosphatidylserine, phosphatidylglycerol, dioleoylphosphatidylglycerol, phosphatidic acid, phosphatidylinositol, sphingomyelin, brain sphingomyelin, cerebrosides, cardiolipins and combinations thereof.

The saturated lipid can be any suitable saturated lipid. More specifically, exemplary saturated phospholipids for use in the lipophilic active agent-encapsulated liposomes of the present invention include hydrogenated soya or egg lecithin, hydrogenated phosphatidylcholine, dilaurylphosphatidylcholine, dimyristoylphosphatidylcholine, distearoylphophatidylcholine, dipalmitoylphosphatidylcholine, 1-myristoyl-2-palmitoylphosphatidylcholine, 1-palmitoyl-2-myristoylphosphatidylcholine, 1-palmitoylphosphatidylcholine, 1-stearoyl-2-palmitoylphosphatidylcholine, hydrogenated phosphatidylethanolamine, dimyristoylphosphatidylethanolamine, dipalmitoylphosphatidylethanolamine, dimyristoylphosphatidylserine, dipalmitoylphosphatidylserine, hydrogenated phosphatidylglycerol, dilauroylphosphatidylglycerol, dimyristoylphosphatidylglycerol, distearoylphosphatidylglycerol, dipalmitoylphosphatidylglycerol, dimyristoylphosphatidic acid, distearoylphosphatidic acid, dipalmitoylphosphatidic acid, hydrogenated phosphatidylinositol, distearoylsphingomyelin, dipalmitoylsphingomyelin, and combinations thereof.

The liposomal composition of the present invention can contain any suitable amount of the at least one membrane forming lipid. In one embodiment, the lipophilic portion can comprise from about 60% to about 100% of the at least one membrane forming lipid. Preferably, the lipophilic portion can comprise from about 65% to about 99% of the at least one membrane forming lipid. More preferably, the lipophilic portion can comprise from about 70% to about 99% of the at least one membrane forming lipid.

In certain embodiments, the protein-assisted active agent-encapsulated liposomes of the present invention can optionally comprise one or more membrane stabilizing agents. Membrane stabilizing agents can be employed in the liposomal composition to ensure stability of the membrane bilayer as well as for retention of drugs incorporated inside the liposomes. The membrane stabilizing agent can be any suitable membrane stabilizing agent. Exemplary membrane stabilizing agents include compounds in the steroid class. In one embodiment, the membrane stabilizing agent is a sterol, sterol derivative, or sterol salt and is preferably cholesterol, coprostanol, cholestanol, cholestane, campesterol, sitosterol, stigmasterol, ergosterol, or combinations, derivatives, or salts thereof. More preferably, the membrane stabilizing agent is cholesterol.

The liposomal composition of the present invention can contain any suitable amount of the membrane stabilizing agent. In one embodiment, the lipophilic portion can comprise from about 0.01% to about 20% of the membrane stabilizing agent. Preferably, the lipophilic portion can comprise from about 0.05% to about 15% of the membrane stabilizing agent. More preferably, the lipophilic portion can comprise from about 0.1% to about 10% of the membrane stabilizing agent.

One of ordinary skill in the art will appreciate that certain membrane forming lipids may contain trace amounts of antioxidants. However, in certain embodiments of the present invention, it may be desirable to add an additional amount of an antioxidant to the liposomal composition. Thus, in one embodiment of the present invention, the lipophilic active agent-encapsulated liposomes of the present invention can optionally comprise one or more added antioxidants to inhibit lipid oxidation.

When an added antioxidant is present, the antioxidant can be any suitable hydrophilic, hydrophobic, or lipophilic antioxidant added to the composition in addition to any residual antioxidant present from the membrane forming lipid. For example, the added antioxidant can be butylated hydroxyanisole, glutathione, propyl gallate, L-ascorbate, alpha-tocopherol, beta-carotene, lycopene, lutein, zeaxanthine, citrates, phosphonates, ethylenediaminetetraacetic acid (EDTA), ascorbic acid, acetylcysteine, sulfurous acid salts, monothioglycerol, and derivatives, salts, or combinations thereof.

When an added hydrophobic or lipophilic antioxidant is present, the liposomal composition of the present invention can contain any suitable amount of the antioxidant. In one embodiment, the lipophilic portion can comprise from about 0.01% to about 20% of the antioxidant. Preferably, the lipophilic portion can comprise from about 0.05% to about 15% of the antioxidant. More preferably, the lipophilic portion can comprise from about 0.1% to about 10% of the antioxidant.

When an added hydrophilic antioxidant is present, the liposomal composition of the present invention can contain any suitable amount of the added antioxidant. In one embodiment, the aqueous portion can comprise from about 0.01% to about 20% of the added antioxidant. Preferably, the aqueous portion can comprise from about 0.05% to about 15% of the added antioxidant. More preferably, the aqueous portion can comprise from about 0.1% to about 10% of the added antioxidant.

In another embodiment, the protein-assisted active agent-encapsulated liposomes of the present invention are substantially free of any added antioxidant. In other words, the only antioxidant present in the composition is the residual antioxidant present from the membrane forming lipid. Preferably, the liposomal composition is substantially free of an added antioxidant when the membrane forming lipid is a saturated phospholipid.

The protein-assisted active agent-encapsulated liposomes of the present invention can optionally be modified so as to further assist in the delivery of the active agent to the BBB. For example, the liposomes can be modified to avoid detection by the body's immune system, specifically, the cells of the reticulo-endothelium system (RES). The RES surveys all antigens or particles entering or circulating in the body and can mobilize an immune response against any article perceived as foreign. In one embodiment, the active agent-encapsulated liposomes of the present invention are modified via conjugation with a ganglioside or polyethylene glycol (PEG). Active agent-encapsulated liposomes modified in this manner are biocompatible, inert, and are characterized by a long half-life in the plasma compartment in vivo. The ganglioside or PEG can be any suitable ganglioside or PEG. For example, the ganglioside can be a monosialoganglioside GM1 derived from bovine brain. Exemplary PEG modifications include liposomal conjugation with PEG-2000 (PEG with average molecular weight of 2000) or PEG-5000 (PEG with average molecular weight of 5000).

Adjusting the length of the PEG to which the liposomes of the present invention are conjugated enables the fine-tuning of plasma half-life of the active agent. In a preferred embodiment, the protein-assisted active agent-encapsulated liposomes of the present invention are modified by PEG conjugation with a phosphatidylethanolamine lipid or a sterol.

In the case of PEG modification of the liposomes of the present invention, a suitable ratio of PEGylated lipid or sterol will be used. In one embodiment, the ratio of PEGylated lipid or sterol can be from about 1% to about 20% of the phospholipid content. Preferably, the ratio of PEGylated lipid or sterol coating can be from about 3% to about 15% of the phospholipid content. More preferably, the ratio of PEGylated lipid or sterol can be from about 5% to about 10% of the phospholipid content.

The liposomes of the present invention may have any suitable zeta potential. In one embodiment, the liposomes have a zeta potential from about +150 mV to about −150 mV.

The active agent-encapsulated liposomes of the present invention can be produced by any suitable method known in the art. The chosen method will depend on the nature of the active agent and the components of the liposomal composition. Liposome preparation typically involves dissolving or dispersing the lipophilic portion (including any lipophilic active agents) in one or more suitable solvents followed by drying. Suitable solvents include any non-polar or slightly polar solvent, such as t-butanol, ethanol, methanol, cyclohexane, chloroform, methylene chloride, or acetone, which can be evaporated without leaving a pharmaceutically unacceptable residue. The drying can be by any suitable means such as rotavapor, thin film agitation, or lyophilization.

Liposomes are then formed when the dried lipid films or lipid cakes are hydrated with a polar, hydrophilic solution, preferably an aqueous solution. Suitable solutions include water or aqueous solutions containing pharmaceutically acceptable salts, buffers, or mixtures thereof. The liposomes are hydrated by dispersing the lipid in the aqueous solution with vigorous mixing or agitation. Any method of mixing or agitation can be used provided that the chosen method induces sufficient shearing forces between the lipid film and polar solvent to strongly homogenize the mixture and form the desired vesicles. Where multilamellar liposomes with highly variable sizes are desired, vortexing or magnetic stiffing may be sufficient. Where unilamellar liposomes or liposomes of a more defined size range are desired, a sonication, filtration, or extrusion step is included in the process.

Sonication can be performed by using, for example, a water bath sonicator (e.g., Branson). The resulting suspension may be subjected to multiple sonication cycles depending upon the size range desired. Alternatively, extrusion may be carried out using a biomembrane extruder such as the Lipex Biomembrane Extruder. Defined pore size in the extrusion filters can generate unilamellar liposomal vesicles of specific sizes. The liposomes of the present invention may also be formed by extrusion through an asymmetric ceramic filter, such as a Ceraflow Microfilter, commercially available from the Norton Company, Worcester Mass.

The size of the active agent-encapsulated liposomes of the present invention can be determined by any suitable method. For example, a particle size analyzer (e.g., Horiba, Malvern, Agilent, or Beckman) can be employed.

Active agent-encapsulated liposomes prepared according to the methods described above can be stored for substantial periods of time prior to administration to a patient. In particular, the liposomes can be produced, sized, and then dehydrated, stored, and subsequently rehydrated for administration. Dehydration can be accomplished by using standard freeze-drying/lyophilization techniques. Liposomes can also be frozen and stored in liquid nitrogen. Additionally, cryoprotectants such as sugars can be added to the buffer during liposome preparation to increase the integrity of the liposome during the dehydration process. In this regard, the active agent-encapsulated liposomes of the present invention are characterized by stability and shelf-lives of several months to several years when lyophilization is employed.

The at least one fimbrial adhesin protein and/or membrane protein can be incorporated into the inventive composition in any suitable manner. When the composition for targeted drug delivery of the present invention is a liposomal composition, the protein can be coated on, bound to, or incorporated in the lipophilic portion of the liposomal composition. For example, a fimbrial adhesin protein may be coated onto liposome particles or covalently bound (grafted) onto the surface of the particle via suitable linking groups to which the protein may be subsequently attached. The fimbrial adhesin protein may also be attached to the surface of active agent-containing liposomes after their preparation by adsorption techniques known to those of skill in the art (e.g., hydrophobic region of peptide to hydrophobic surface of a suitable particle, etc.).

Alternatively, when the composition of the present invention is a liposomal composition further modified with a ganglioside or PEG, the fimbrial adhesin protein can be bound to the ganglioside or PEG. In this regard, the fimbrial adhesin protein can be bound to the ganglioside or PEG by in any suitable manner. In a preferred embodiment, the fimbrial adhesin protein is bound to a PEG which is conjugated to the active agent-encapsulated liposomes.

In yet another embodiment, when the composition of the present invention is a liposomal composition, the at least one fimbrial adhesin protein and/or membrane protein can be incorporated into the composition via a bond to the active agent. The protein can be bound to the active agent in any suitable manner. For example, the protein can be covalently or non-covalently bound to the active agent. Wherein the protein is covalently bound to the active agent, the binding can occur between any suitable functional groups on the protein and the active agent. Covalent bonding may also occur via any suitable linking or spacing groups to which the protein and active agent may be subsequently attached. The active agent may be bound to the protein directly or via bi-functional linkers/spacers. Wherein the protein is non-covalently bound to the active agent, the binding can occur via any suitable non-covalent means. Exemplary non-covalent interactions include ionic/electrostatic bonds, hydrophobic interactions, hydrogen bonds, Van der Waals forces (i.e., London dispersion forces), and dipole-dipole bonds.

In this regard, the at least one fimbrial adhesin protein and/or membrane protein or variant thereof may be used as a macromolecular carrier wherein the active agent is attached to the protein molecule directly and is not necessarily encapsulated within a liposome. As such, the present invention is also directed to a prodrug for targeted drug delivery to the CNS of a patient comprising an active agent bound to at least one fimbrial adhesin protein and/or membrane protein or variant thereof and methods for the preparation of the prodrug. As set forth above, protein can be bound to the active agent in any suitable manner. The prodrug of the present invention is capable of selectively targeting and penetrating the BBB. Once across the BBB, the prodrug is metabolized and converted to the parent active agent. The active agent for use in the inventive prodrug can be any suitable active agent set forth above provided the active agent contains a functional group capable of forming a covalent bond with a fimbrial adhesin protein and/or membrane protein or variant thereof.

In another embodiment, the present invention provides a composition for targeted drug delivery to the CNS of a patient comprises a pharmaceutically active agent bound to at least one protein selected from the group consisting of a fimbrial adhesion protein, a membrane protein, and combinations thereof, and further comprises (i) a bi-functional linker or spacer between the pharmaceutically active agent and the at least one protein. Preferably, the composition further comprises a pharmaceutically acceptable carrier that is a pH stabilized solution wherein the pH of the solution is in a range from about 2 to about 9.

The pH stabilized solution may comprise any suitable buffering agent that maintains the pH of the solution in a range from about 2 to about 9. For example, the buffering agent may be selected from the group consisting of acetic acid, citric acid, glyoxalic acid, glutamic acid, lactic acid, alanine, maleic acid, crotonic acid, succinic acid, tartaric acid, piperazine, itaconic acid, glutaric acid, histamine, ascorbic acid, gallic acid, phosphoric acid and salts thereof.

The pH stabilized solution may further comprise any suitable antioxidant. The antioxidant may be any of the antioxidants described herein.

The at least one protein for use in the prodrug of the present invention can be any suitable protein or variant thereof set forth above. In a preferred embodiment of the present invention, the fimbrial adhesin protein is S fimbriae isolated from E. coli K1, E. coli K1-subtypes or E. coli CFT073. In another preferred embodiment of the present invention, the membrane protein is OmpA isolated from E. coli K1, E. coli K1-subtypes or E. coli 0157:H7. In yet another preferred embodiment, the prodrug of the present invention comprises a combination of S fimbriae isolated from E. coli K1, E. coli K1-subtypes or E. coli CFT073 and OmpA isolated from E. coli K1, E. coli K1-subtypes or E. coli 0157:H7.

The present invention further provides for methods of delivering a pharmaceutically acceptable active agent to the CNS of a patient in need thereof using the targeted drug delivery compositions described above. In one embodiment, the present invention provides a method of delivering an active agent to the brain of a patient in need thereof. In another embodiment, the inventive method is directed to delivering an active agent to the spinal cord of a patient in need thereof.

In one embodiment, a method of delivering a pharmaceutically acceptable active agent to a patient in need thereof comprises administering to the patient a composition comprising liposomes containing (i) an encapsulated pharmaceutically active agent, (ii) at least one membrane forming lipid, wherein at least a portion of the lipid is derivatized by a hydrophilic polymer to form a hydrophilic polymer-derivatized lipid and at least a portion of the hydrophilic polymer-derivatized lipid is functionalized to attach a targeting ligand, and (iii) at least one membrane stabilizing agent. The composition comprising liposomes is as described herein.

In one embodiment, a method of delivering a pH stabilized solution of a pharmaceutically active agent to a CNS of a patient via the bloodstream comprises administering to the patient a pH stabilized solution comprising (i) a pharmaceutically active agent bound to at least one protein selected from the group consisting of a fimbrial adhesion protein, a membrane protein, and combinations thereof, and (ii) a buffering agent, wherein the pH of the solution is from about 2 to about 9. The pH stabilized solution of a pharmaceutically active agent is as described herein.

The present invention provides a method of delivering an active agent to a patient in need of any CNS related therapy. For example, the patient can be in need of treatment for Alzheimer's disease, Parkinson's disease, brain cancer, stroke, brain injury, spinal cord injury, HIV infection of the brain, an ataxia-producing disorder, amyotrophic lateral sclerosis, Huntington disease, multiple sclerosis, affective disorders, anxiety disorders, epilepsy, meningitis, neuromyelitis optica, late-stage neurological trypanosomiasis, progressive multifocal leukoencephalopathy, De Vivo disease, depression, chronic pain, or a childhood inborn genetic error affecting the brain. In additional embodiments, the patient can be in need of treatment with antineoplastics, antidepressants, anti-inflammatory, antipsychotics, analgesics, or sedatives.

In accordance with the inventive method, the targeted drug delivery compositions can be formulated for any suitable means of administration known in the art. For example, the composition can be administered transdermally, intraperitoneally, intracardially, intramuscularly, locally, orally, intravenously, or subcutaneously.

The following examples further illustrate the invention but, of course, should not be construed as in any way limiting its scope.

### Example 1

This example demonstrates a method of making a fimbrial adhesion protein or a membrane protein.

Cloning of Proteins from E. coli

Three E. coli proteins, sfaS, sfaA, and ompA, are cloned into the plasmid, pET22b. First, the genes are PCR amplified from E. coli to contain the sequences SEQ ID NO: 1 (corresponding to sfaS from E. coli CFT073, with the first (5′) 22 amino acids removed and “atg” added to the 5′ end), SEQ ID NO: 3 (corresponding to sfaA from E. coli CFT073, with the first (5′) 30 amino acids removed and “atg” added to 5′ end) and SEQ ID NO: 5 (ompA from E. coli 0157:H7). The genes are then ligated with pET22b (sfaA and sfaS) and PET21a (ompA) to contain a C-terminal His-tag and transformed into E. coli BL21 (DE3). The sequences of the clones are confirmed by DNA sequence analysis.

### Protein Purification

Appropriate media (2.0 ml, containing antibiotic) is inoculated in a culture tube with a single colony from a plate. Colonies are incubated at 37° C. with shaking at 250 rpm to an OD600 of approximately 1.0. Further, the entire 2.0 ml culture is added to 20 ml medium containing antibiotics. The culture is incubated at 37° C. with shaking at 250 rpm to an OD600 of approximately 1.0. Further, the entire 20 ml culture is added to 2000 ml medium containing antibiotics. The culture is shaken at the desired temperature until the OD600 is approximately 0.6 (e.g., 3.5 h in LB broth, 37° C.). The OD600 is monitored during growth by removing aliquots aseptically. Finally, IPTG (Isopropyl-beta-D-thiogalactopyranoside, from Amerisco) is added to the cultures for a final concentration of 1 mM. Both cultures are incubated with shaking at 37° C. for 4 h, as appropriate.

After the induced cells grow for the proper length of time, the cells are harvested by centrifugation at 3500 g for 15 minutes at 4° C. Cells are washed with 100 ml PBS. The sample is centrifuged at 3500 g for 15 min at 4° C. The supernatant is discarded, and the cell pellet is frozen and stored overnight at −20° C. The cell pellet is thawed for 15 min on ice and resuspended in column binding buffer (100 mM NaH₂PO₄; 10 mM Tris.Cl; 6M GuHCl; pH8.0) at 5 ml per gram wet weight. Cells are stirred for 15-60 min at room temperature or lysed by gently vortexing; taking care to avoid foaming. Lysis is complete when the solution becomes translucent. Lysate is centrifuged at 12,000 g for 30 min at room temperature to pellet the cellular debris. The supernatant is saved for future use. SDS-PAGE sample buffer (5 μl 2×) is added to 5 μl supernatant and stored at −20° C. for SDS-PAGE analysis.

The bottle of the resin is gently inverted to mix the slurry, and 2 ml is transferred to a 2.5×10 cm glass column. The resin is allowed to pack under gravity flow. The resin is washed with 3 column volumes of sterile H₂O. The resin is equilibrated with 6 column volumes of column binding buffer. The column binding buffer above the resin is allowed to drain to the top of the column. The cell lysate is immediately loaded onto the column. The flow rate is adjusted to 1 ml/minute. Unbound proteins are washed from the resin by adding 10 bed volumes of column binding buffer. The column is washed with 6 column volumes of column wash buffer (100 mM NaH₂PO₄; 10 mM Tris.Cl; 8 M urea; pH 6.3). Continue washing the column until the A280 of the flow through is <0.01.

After the column with bound protein is washed and drained, the column outlet is closed. The bound fusion protein is eluted by adding 1 ml of column elution buffer (100 mM NaH₂PO₄; 10 mM Tris.Cl; 8 M urea; pH 4.5). The column is incubated at room temperature for 10 min to elute the fusion protein. The column outlet is opened and the eluate is collected. The elution and collection steps are repeated twice more, pooling all three eluates and storing them at −70° C. The fusion protein is assayed by analyzing 20-μl aliquots by electrophoresis through a 12% SDS-polyacrylamide gel.

### Characterization of Proteins

The purity of the proteins is determined by 10% SDS-PAGE analysis and stained with coomassie blue for total protein. The identities of the proteins are confirmed using MALDI-Mass spectrometry analysis. The estimations of purity and molecular weights as measured by SDS-PAGE and the amino acid sequences of the proteins are set forth in Table 1.

TABLE 1

| | Amino | Estimation of | Molecular |
|---|---|---|---|
| Name | AcidSequence | Purity | Weight |
| SfaS | SEQ ID NO: 2 | 90% | 17.8 kD |
| SfaA | SEQ ID NO: 4 | 95% | 19.7 kD |
| OmpA | SEQ ID NO: 6 | 80% | 39.6 kD |

This example demonstrated a method of making a fimbrial adhesion protein (SfaA or SfaS) or a membrane protein (OmpA).

### Example 2

This example demonstrates that a fimbrial adhesion protein binds to bovine brain microvascular endothelial cells (BBMVECs). This example also demonstrates that a membrane protein binds to bovine brain microvascular endothelial cells (BBMVECs).

The three E. coli recombinant proteins (SfaS, SfaA, OmpA) produced in Example 1 are labeled with fluorescein isothiocyanate (FITC) to allow fluorescent detection of proteins. The proteins are labeled using the Thermo-Fisher Pierce FITC labeling kit (Thermo Fisher Scientific Inc., Waltham, Mass.) according to the manufacturer's protocol. The amounts of protein used for the labeling reaction are 0.75 mg (SfaS), 0.8 mg (OmpA) and 1.25 mg (SfaA).

Flow cytometry is performed by incubating the labeled proteins with BBMVECs. These cells are procured from Cell Applications, Inc (San Diego, Calif.). Cells are mixed with FITC proteins (individually) and incubated on ice or at 37° C. for 15 minutes, then washed, and sorted with flow cytometry.

A significant shift in fluorescence is observed for both OmpA-FITC and SfaA-FITC at 4° C. A less significant shift is observed for SfaS. For OmpA, ˜17% of the cells strongly bind to the protein and for SfaA, ˜37% of the cells bind. When the cells are incubated at 37° C., there is a significantly smaller percentage of cells that bind the proteins. For OmpA, ˜9% of the cells bind strongly and for SfaA, ˜10% of the cells bind. It is believed that lower fluorescence binding of OmpA-FITC and SfaA-FITC at 37° C. compared to the 4° C. may be due to the internalization of bound proteins at 37° C., and that internalization of the proteins may result in a decrease fluorescence signal.

This example demonstrated that both OmpA and SfaA proteins from E. coli bind to the bovine brain microvascular endothelial cells (BBMVECs) at 4° C., and that both proteins facilitate the transport of drugs or drug delivery carriers through the blood brain barrier.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition for targeted drug delivery to the central nervous system of a patient comprising
a. at least one protein selected from the group consisting of a fimbrial adhesion protein, a membrane protein, and combinations thereof;
b. a pharmaceutically acceptable active agent; and
c. a pharmaceutically acceptable carrier.

2. The composition of claim 1, wherein the at least one protein is a fimbrial adhesion protein selected from the group consisting of S fimbriae, variants of S fimbriae, and combinations thereof.

3. The composition of claim 2, wherein the fimbrial adhesion protein comprises S fimbriae or a variant of S fimbriae isolated from Escherichia coli K1, Escherichia coli K1-subtypes or Escherichia coli CFT073.

4. The composition of claim 2, wherein the fimbrial adhesion protein is a variant of S fimbriae comprising a polypeptide fragment of S fimbriae which maintains the functional domain or domains of S fimbriae involved in CNS delivery.

5. The composition of claim 1, wherein the at least one protein is a membrane protein selected from the group consisting of outer membrane protein A, variants of outer membrane protein A (OmpA), and combinations thereof.

6. The composition of claim 5, wherein the membrane protein is an outer membrane protein A or a variant of outer membrane protein A isolated from Escherichia coli K1, Escherichia coli K1-subtypes or Escherichia coli 0157:H7.

7. The composition of claim 1, wherein the pharmaceutically active agent is bound to the at least one protein, and the composition further comprises (i) a bi-functional linker or spacer between the pharmaceutically active agent and the at least one protein.

8. The composition of claim 2, wherein fimbrial adhesion protein is a variant of S fimbriae comprising SEQ ID NO: 2 or 4.

9. The composition of claim 5, wherein the membrane protein is an outer membrane protein A comprising SEQ ID NO: 6.

10. A liposomal composition for targeted drug delivery to the central nervous system of a patient comprising
a. a targeting ligand comprising at least one protein selected from the group consisting of a fimbrial adhesion protein, a membrane protein, and combinations thereof;
b. a pharmaceutically acceptable active agent; and
c. liposomes comprising (i) a membrane forming lipid wherein a portion of the lipid is derivatized by a hydrophilic polymer to form hydrophilic polymer-derivatized lipid and at least a portion of the hydrophilic polymer-derivatized lipid is functionalized to attach to the targeting ligand, and (ii) a membrane stabilizing agent.

11. The liposomal composition of claim 10, wherein the hydrophilic polymer is selected from the group consisting of polyethylene glycol (PEG), polyvinylpyrrolidone (PVP), polyvinylmethylether, polymethyloxazoline, polyethyloxazoline, polyhydroxypropyloxazoline, polymethacrylamide, polydimethacrylamide, polyhydroxypropylmethacrylamide, polyhydroxypropylmethacrylate, polyhydroxyethylacrylate, hydroxymethylcellulose, hydroxyethylcellulose, polyaspartamide, and polysaccharide.

12. The liposomal composition of claim 11, wherein the targeting ligand is attached to a distal end of the functionalized hydrophilic polymer-derivatized lipid having a compound of the formula:
membrane forming lipid-PEG-X
wherein X is a functional group selected from the group consisting of —OH, —CHO, —COOH, —SH, —NHS, —NHCO, —NHCS, —NH₂, -maleimide, -isocyanate, -hydrazide, -vinylsulfone, and -epoxide.

13. The liposomal composition of claim 12, wherein the targeting ligand is derivatized to generate a functional group selected from the group consisting of —OH, —NH₂, —SH, and —COOH.

14. The liposomal composition of claim 10, wherein the at least one protein is a fimbrial adhesion protein selected from the group consisting of S fimbriae, variants of S fimbriae, and combinations thereof.

15. The liposomal composition of claim 10, wherein the at least one protein is a membrane protein selected from the group consisting of outer membrane protein A, variants of outer membrane protein A, and combinations thereof.

16. A method of delivering a pharmaceutically acceptable active agent to the central nervous system of a patient in need thereof, comprising administering to the patient a composition comprising liposomes containing (i) an encapsulated pharmaceutically active agent; (ii) at least one membrane forming lipid, wherein a portion of the lipid is derivatized by a hydrophilic polymer to form a hydrophilic polymer-derivatized lipid and at least a portion of the hydrophilic polymer-derivatized lipid is functionalized to attach a targeting ligand; and (iii) at least one membrane stabilizing agent.

17. The method of claim 16, wherein the membrane forming lipid is a cationic lipid effective to impart a positive surface charge to the liposomes, and wherein the positive surface charge enhances binding of liposomes to target cells.

18. The method of claim 16, wherein the liposomes contain a targeting ligand attached to a distal end of the functionalized hydrophilic polymer-derivatized lipid, wherein the targeting ligand is a protein selected from the group consisting of a fimbrial adhesion protein, a membrane protein, and combinations thereof.

19. The method of claim 18, wherein the protein is a fimbrial adhesion protein selected from the group consisting of S fimbriae, variants of S fimbriae, and combinations thereof.

20. The method of claim 18, wherein the protein is a membrane protein selected from the group consisting of outer membrane protein A, variants of outer membrane protein A, and combinations thereof.

21. The method of claim 19, wherein the protein is a variant of S fimbriae comprising SEQ ID NO: 2 or 4.

22. The method of claim 20, wherein the protein is an outer membrane protein A comprising SEQ ID NO: 6.

23. A method of delivering a pH stabilized solution of a pharmaceutically active agent to a CNS of a patient via the bloodstream comprising administering to the patient a pH stabilized solution comprising (i) a pharmaceutically active agent bound to at least one protein selected from the group consisting of a fimbrial adhesion protein, a membrane protein, and combinations thereof; and (ii) a buffering agent, wherein the pH of the solution is from about 2 to about 9.

24. The method of claim 23, wherein the pharmaceutically active agent is bound to the protein directly or via bi-functional linkers/spacers.

25. The method of claim 23, wherein the pH stabilized solution further contains an anti-oxidant selected from the group consisting of alpha-tocopherol, ascorbic acid, acetylcysteine, sulfurous acid salts, monothioglycerol, EDTA and derivatives or salts thereof.

26. The method of claim 23, wherein the protein is a fimbrial adhesion protein selected from the group consisting of S fimbriae, variants of S fimbriae, and combinations thereof.

27. The method of claim 23, wherein the protein is a membrane adhesion protein selected from the group consisting of outer membrane protein A, variants of outer membrane protein A, and combinations thereof.

28. The method of claim 26, wherein the protein is a variant of S fimbriae comprising SEQ ID NO: 2 or 4.

29. The method of claim 27, wherein the protein is an outer membrane protein A comprising SEQ ID NO: 6.


SEQUENCE LISTING (6 sequences)

SEQ ID NO: 1 - DNA, 441 bp, E. coli
atggtggatacgacgattactgttacagggagggtattgccacgtacctgtaccattggtaatggaggaaacccaaacgccaccgttgttttggataacgcttacacttctgacctgatagcagccaacagcacctctcagtggaaaaatttttcgttgacattgacgaattgtcagaatgtaaacaatgttactgccacctttggtggaaccgcagaaaatacaaattattacagaaatacaggggatgctactaatatcatggttgagctacaggaacaaggtaatggtaatacccccttgaaagttggttcaacaaaagttgttacagtgagcaatgggcaggcgacattcaatcttaaagtccgtgccgtaagcaaaggtaatgctggtgcgggaagtattaattcacaaattactgtcacctatacctatgcgtaa

SEQ ID NO: 2 - protein, 168 aa, E. coli
MASMTGGQQMGRGSMVDTTITVTGRVLPRTCTIGNGGNPNATVVLDNAYTSDLIAANSTSQWKNFSLTLTNCQNVNNVTATFGGTAENTNYYRNTGDATNIMVELQEQGNGNTPLKVGSTKVVTVSNGQATFNLKVRAVSKGNAGAGSINSQITVTYTYALEHHHHHH

SEQ ID NO: 3 - DNA, 456 bp, E. coli
atgggtacagttcattttaagggtgaagtggttaatgctgcatgtgctgtaaacactaactcattcgatcagacggttaatcttggacaggttcgttccgaaagattgaaagtagatggagctaaaagcaatccagttggatttacaattgaattaaatgattgtgactcgcaggtgtctgctggtgcaggaattgtcttttcaggcccagcagttactggtaaaacagatgttcttgctttacaaagttctgcagcgggttctgcaacaaacgtcggcgttcagattactgaccataccggcaaggttgtacctttagatggaactgcaagctcaacgtttacattaactgacggaaccaacaaaattccatttcaggcggtttactacgcaactggacaggccactgctggtattgccaacgccgacgccacctttaaagttcagtaccagtaa

SEQ ID NO: 4 - protein, 190 aa, E. coli
MKYLLPTAAAGLLLLAAQPAMAMDIGINGTGSMGTVHFKGEVVNAACAVNTNSFDQTVNLGQVRSERLKVDGAKSNPVGFTIELNDCDSQVSAGAGIVFSGPAVTGKTDVLALQSSAAGSATNVGVQTDHTGKVVPLDGTASSTFTLTDGTNKIPFQAVYYATGQATAGIANADATFKVQYQLEHHHHHH

SEQ ID NO: 5 - DNA, 1041 bp, E. coli
atgaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcatgctgagcctgggtgtttcctaccgtttcggtcagggcgaagcagctccagtagttgctccggctccagctccggcaccggaagtacagaccaagcacttcactctgaagtctgacgttctgttcaacttcaacaaagcaaccctgaaaccggaaggtcaggctgctctggatcagctgtacagccagctgagcaacctggatccgaaagacggttccgtagttgttctgggttacaccgaccgcatcggttctgacgcttacaaccagggtctgtccgagcgccgtgctcagtctgttgttgattacctgatctccaaaggtatcccggcagacaagatctccgcacgtggtatgggcgaatccaacccggttactggcaacacctgtgacaacgtgaaacagcgtgctgcactgatcgactgcctggctccggatcgtcgcgtagagatcgaagttaaaggtatcaaagacgttgtaactcagccgcaggcttaa

SEQ ID NO: 6 - protein, 366 aa, E. coli
MASMTGGQQMGRGSMKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDYTRLGGMVWRADTKSNVYGKNHDTGVSPVFAGGVEYAITPEIATRLEYQWTNNIGDAHTIGTRPDNGMLSLGVSYRFGQGEAAPVVAPAPAPAPEVQTKHFTLKSDVLFNFNKATLKPEGQAALDQLSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQALEHHHHHH